(19) United States
(12) Patent Application Publication
(10) Pub. No.: US 2004/0009592 A1
(43) Pub. Date: Jan. 15, 2004

(54) Genetically-modified neural progenitors and uses thereof

(75) Inventors: Olivier Sabate, Paris (FR); Philippe Horellou, Paris (FR); Marie-Helene Buc-Caron, Paris (FR); Jacques Mallet, Paris (FR)

Correspondence Address: FINNEGAN, HENDERSON, FARABOW, GARRETT & DUNNER, LLP, 1300 I STREET, NW, WASHINGTON, DC, 20005, US

(73) Assignee: Rhone-Poulenc Rorer S.A.

(21) Appl. No.: 10/305,386

(22) Filed: Nov. 27, 2002

Related U.S. Application Data
(63) Continuation of application No. 08/810,315, filed on Feb. 28, 1997, now abandoned.
(60) Provisional application No. 60/012,635, filed on Mar. 1, 1996.

(51) Int. Cl. C12N 5/08

(52) U.S. Cl. 435/368

(57) ABSTRACT

The invention concerns human neural progenitor cells containing introduced genetic material encoding a product of interest, and their use for the treatment of neurodegenerative diseases.

### FIELD OF THE INVENTION

This invention concerns the domain of neurobiology. More specifically, it relates to genetically-modified neural progenitor cells having therapeutic properties and their use, especially for the treatment of neurodegenerative disorders.

### BACKGROUND OF THE INVENTION

Development of an efficient therapy for neurodegenerative disorders, such as Parkinson's, Huntington's or Alzheimer's disease, represents an important clinical challenge. Pioneering studies of neural grafts in rodents have indicated great potential for restorative therapy (for review, see ref 1). Grafts of human fetal brain tissue are currently under clinical investigation for patients with Parkinson's disease² and proposed for other neurodegenerative diseases such as Huntington's disease. However, these encouraging preliminary studies raise important questions for potential extension to larger numbers of patients. In particular, practical and ethical problems will arise from the growing use of human fetuses for clinical purpose. Technical improvements should be performed in cryopreservation and increasing the number of donor cells from a single human embryo before this method could be widely used³. Thus, generalized clinical use of human fetal tissue is problematic, not only from an ethical point of view but also because of the limited supply.

The selective growth of neural progenitor cells provides a way of circumventing these problems. Recently, the generation of neurons and astrocytes from precursors maintained in a state of proliferation with EGF⁵ or long-term cultures of neuroblasts in presence of bFGF⁶,⁷ may provide large amounts of cells for brain repair. Nevertheless, large numbers of appropriately differentiated cells, such as dopaminergic cells, may be difficult to obtain. In addition, therapeutic use of neuronal cells have been impeded by the lack of availability of competent human cells, and the lack of efficient means to modify, amplify and use such cells. Finally, it is unclear whether or not human competent cells can be used clinically.

### SUMMARY OF THE INVENTION

The present invention describes a human neural progenitor cell comprising an exogenous nucleic acid encoding a neuroactive substance. In a preferred embodiment, the human neural progenitor cell is a neuroepithelial stem cell.

In one aspect of the invention the neuroactive substance is a DNA encoding a protein or peptide. Preferred proteins or peptides include growth factors, neurotrophic factors and enzymes.

In another aspect of the invention the neuroactive substance is a DNA encoding an antisense-RNA or a ribozyme.

A preferred aspect of the invention is a human neural progenitor cell comprising an exogenous nucleic acid encoding a neuroactive substance, wherein said nucleic acid has been introduced into said cell with a viral vector. In a most preferred aspect of the invention, the viral vector is a replication defective adenovirus.

The nucleic acid encoding a neuroactive substance may be operably linked to a regulatory region. Preferably, the regulatory region comprises a regulatable promoter, an inducible promoter, a neural cell-specific promoter or a viral promoter., The present invention also provides an implant comprising a human neural progenitor cell comprising an exogenous nucleic acid encoding a neuroactive substance.

Another aspect of the invention is a composition comprising a human neural progenitor cell comprising an exogenous nucleic acid encoding a neuroactive substance. The compostion may additionally comprise neuroblasts or glial precursors comprising an exogenous nucleic acid encoding a neuroactive substance.

### BRIEF DESCRIPTION OF THE DRAWINGS

FIG. 1: In vitro transfer of the Lac Z gene into human neural progenitors using recombinant adenoviral vectors. Primary cultures of neural progenitors from a human fetus (8 weeks of gestation) were grown for 7 DIV and infected with Ad-RSVβGal (MOI=500). Five days after infection, X-gal staining revealed blue nuclei, indicative of expression of the Lac Z gene, nuclearly targeted by the SV40 nuclear localisation signal. Cell types were identified by immunocytochemistry using primary specific and fluorescence conjugated antibodies. Arrows point to double labeled cells in phase contrast photomicrographs (A,C,E,G) and their corresponding fluorescence photomicrographs (B,D,F,H), providing clear evidence for expression of the transgene in the different cell types of neural progenitors. Neuroepithelial stem cells are identified by their epithelial shape (A,C) and their staining with anti-nestin (B) and anti-vimentin (D). Cells already committed to the neuronal lineage show round refringent perikarya and bipolar processes (E) together with the presence of β3-tubulin (F) and MAP5 (not shown). Glial precursors have a flat morphology (G) and are decorated by the monoclonal A2B5 (H) and HNK-1 (not shown). Magnification for all the photomicrographs is 220×.

FIG. 2: β-galactosidase expression in human neuroblasts transplanted to the rat striatum after in vitro infection with recombinant adenoviruses. 3 weeks after grafting of 1×10⁶ human neural progenitors, the rat was sacrificed and 15 μm cryostat sections were obtained. Numerous blue nuclei, corresponding to transplanted cells expressing the transgene, were found after incubation with X-gal (for 3 hours to minimize artifactual staining) (A,B,C,E,F. The specificity of the staining was confirmed by the similar labeling pattern found in adjacent sections after X-gal staining (blue nuclei in C) and after incubation with an antibody specific for E. coli β-galactosidase (brown nuclei in D). X-gal staining followed by incubation with antibodies specific for neuronal and glial lineages identified numerous double labeled neuroblasts (A,B,E,F) harboring a blue nucleus and brown cytoplasm and even in some cases brown processes. The labelling with a human specific NSE confirmed the human nature of the positive cells and their commitment to the neuronal lineage (A,B). Grafted cells were also positive for both the neuronal markers β-tubulin (E) and MAP5 (F). Anti-GFAP (GA5 clone) revealed reactive astrocytes surrounding the grafted cells but no glial cell was found to express β-galactosidase (C). Magnifications were 50 (A), 200 (B) and 100 (C,D,E,F).

### DETAILED DESCRIPTION OF THE INVENTION

Definitions

The following defined terms are used throughout the present specification, and should be helpful in understanding the scope and practice of the present invention.

A “polypeptide” is a polymeric compound comprised of covalently linked amino acid residues. Amino acids have the following general structure:

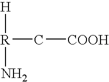

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

A “protein” is a polypeptide which plays a structural or functional role in a living cell.

The polypeptides and proteins of the invention may be glycosylated or unglycosylated.

A “variant” of a polypeptide or protein is any analogue, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. The skilled artisan can produce variants having single or multiple amino acid substitutions, deletions, additions, or replacements. These variants may include, inter alia: (a) variants in which one or more amino acid residues are substituted with conservative or non-conservative amino acids, (b) variants in which one or more amino acids are added to the polypeptide or protein, (c) variants in which one or more of the amino acids includes a substituent group, and (d) variants in which the polypeptide or protein is fused with another polypeptide such as serum albumin. The techniques for obtaining these variants, including genetic (suppressions, deletions, mutations, etc.), chemical, and enzymatic techniques, are known to persons having ordinary skill in the art.

If such allelic variations, analogues, fragments, derivatives, mutants, and modifications, including alternative mRNA splicing forms and alternative post-translational modification forms result in derivatives of the polypeptide which retain any of the biological properties of the polypeptide, they are intended to be included within the scope of this invention.

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA. The sequence of nucleotides that encodes a protein is called the sense sequence. An “exogenous nucleic acid” is genetic material which has been introduced into a cell not naturally containing the nucleic acid sequence.

“Regulatory region” means a nucleic acid sequence which regulates the expression of a second nucleic acid sequence. A regulatory region may include sequences which are naturally responsible for expressing a particular nucleic acid (a homologous region) or may include sequences of a different origin (responsible for expressing different proteins or even synthetic proteins). In particular, the sequences can be sequences of eukaryotic or viral genes or derived sequences which stimulate or repress transcription of a gene in a specific or non-specific manner and in an inducible or non-inducible manner. Regulatory regions include origins of replication, RNA splice sites, enhancers, transcriptional termination sequences, signal sequences which direct the polypeptide into the secretory pathways of the target cell, and promoters.

A regulatory region from a “heterologous source” is a regulatory region which is not naturally associated with the expressed nucleic acid. Included among the heterologous regulatory regions are regulatory regions from a different species, regulatory regions from a different gene, hybrid regulatory sequences, and regulatory sequences which do not occur in nature, but which are designed by one having ordinary skill in the art.

A “vector” is any means for the transfer of a nucleic acid according to the invention into a host cell. The term “vector” includes both viral and nonviral means for introducing the nucleic acid into a cell in vitro, ex vivo or in vivo. Non-viral vectors include plasmids, liposomes, electrically charged lipids (cytofectins), DNA-protein complexes, and biopolymers. Viral vectors include retrovirus, adeno-associated virus, pox, baculovirus, vaccinia, herpes simplex, Epstein-Barr and adenovirus vectors. In addition to a nucleic acid according to the invention, a vector may also contain one or more regulatory regions, and/or selectable markers useful in selecting, measuring, and monitoring nucleic acid transfer results (transfer to which tissues, duration of expression, etc.).

“Pharmaceutically acceptable carrier” includes diluents and fillers which are pharmaceutically acceptable for methods of administration, are sterile, and may be aqueous or oleaginous suspensions formulated using suitable dispersing or wetting agents and suspending agents. The particular pharmaceutically acceptable carrier and the ratio of active compound to carrier are determined by the solubility and chemical properties of the composition, the particular mode of administration, and standard pharmaceutical practice.

Neural Progenitor Cells

One aspect of the instant invention is to provide human neural progenitor cells containing introduced genetic material encoding a product of interest. Another aspect of the instant invention is to provide human neural progenitor cells having desired therapeutic properties, suitable for grafting. Yet another aspect of the instant invention is to provide a composition comprising modified cells wherein said composition comprises at least human neural progenitor cells, possibly associated with neuroblasts and/or glial precursors. Transplantation of genetically modified human neural progenitor cells to rat brain is disclosed in Sabate et al. (Nature Genetics, Volume 9, pp. 256-260 (1995)), the entire contents of which are incorporated herein by reference.

More specifically, neuroepithelial cells have been isolated and characterized from human brain fetuses. Identification of the cells as neural precursors rests upon their labelling with anti-nestin and anti-vimentin antibodies. Furthermore, they differentiate progressively in serum-containing medium into neuronal and glial cells while they proliferate and maintain an immature still plastic phenotype in serum-free defined conditions supplemented with bFGF. In the perspective of generating large amounts of cells expressing a gene of interest from a single human embryo, we have furthermore shown hat it is possible to amplify and confer desired properties to these cells.

The instant invention now provides a very efficient way to obtain high proportion of cells producing factors with biological effect, such as neurotransmitters or growth factors, in the perspective of grafting. The inventors have now found conditions that enable successful amplification, in vitro modification, and grafting of these cells. Substantial levels of expression were obtained in cells of the neuronal and glial lineages in vitro and in vivo in neuroblasts. Thus genetically modifying human precursor cells according to the invention offers great promises for the future of gene therapy in neurodegenerative diseases. In addition, the invention now provides large amounts of progenitor cells of human origin with desired properties, suitable for grafting allowing wider clinical use of transplantations.

In a first aspect, the invention thus concerns human neural progenitor cells containing introduced genetic material encoding a product of interest, such as a neuroactive substance. More specifically, the human neural progenitor cells of the invention comprise an exogenous nucleic acid encoding a neuroactive substance. The human neural progenitor cells are reactive with anti-nestin and anti-vimentin antibodies. In a preferred embodiement, the cells of the invention are derived from human foetal brains. The cells of the invention are more specifically neuroepithelial stem cells.

In an other aspect, the invention concerns a composition comprising modified cells wherein said composition comprises at least human neural progenitor cells containing introduced genetic material encoding a product of interest, such as a neural active substance. In a specific embodiement, the composition of the invention further comprises neuroblasts containing introduced genetic material encoding a product of interest. Neuroblasts can further be characterized by the presence at the cell surface of specific markers such as MAP5 and β3-tubulin. In an other specific embodiement, the composition of the invention further comprises glial precursors containing introduced genetic material encoding a product of interest. Glial precursors can be further characterized by the presence at the cell surface of specific markers such as A2B5 and HNK-1.

Vectors

A preferred way to genetically modify the cells or compositions according to the instant invention employs viral vectors. Viral transduction can be made using several types of viral vectors, including adenovirus, herpes virus, AAV, retrovirus, and vaccinia virus. A more preferred viral vector is an adenovirus-derived vector.

Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents.

Preferably, the replication defective virus retains the sequences of its genome which are necessary for encapsidating the viral particles.

The retroviruses are integrating viruses which infect dividing cells. The retrovirus genome includes two LTRs, an encapsidation sequence and three coding regions (gag, pol and env). The construction of recombinant retroviral vectors has been described: see, in particular, EP 453242, EP178220, Bernstein et al. Genet. Eng. 7 (1985) 235; McCormick, BioTechnology 3 (1985) 689, etc. In recombinant retroviral vectors, the gag, pol and env genes are generally deleted, in whole or in part, and replaced with a heterologous nucleic acid sequence of interest. These vectors can be constructed from different types of retrovirus, such as, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Defective retroviral vectors are disclosed in WO95/02697.

In general, in order to construct recombinant retroviruses containing a nucleic acid sequence, a plasmid is constructed which contains the LTRs, the encapsidation sequence and the coding sequence. This construct is used to transfect a packaging cell line, which cell line is able to supply in trans the retroviral functions which are deficient in the plasmid. In general, the packaging cell lines are thus able to express the gag, pol and env genes. Such packaging cell lines have been described in the prior art, in particular the cell line PA317 (U.S. Pat. No. 4,861,719); the PsiCRIP cell line (WO90/02806) and the GP+envAm-12 cell line (WO89/07150). In addition, the recombinant retroviral vectors can contain modifications within the LTRs for suppressing transcriptional activity as well as extensive encapsidation sequences which may include a part of the gag gene (Bender et al., J. Virol. 61 (1987) 1639). Recombinant retroviral vectors are purified by standard techniques known to those having ordinary skill in the art.

The adeno-associated viruses (AAV) are DNA viruses of relatively small size which can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions which carry the encapsidation functions: the left-hand part of the genome, which contains the rep gene involved in viral replication and expression of the viral genes; and the right-hand part of the genome, which contains the cap gene encoding the capsid proteins of the virus.

The use of vectors derived from the AAVs for transferring genes in vitro and in vivo has been described (see WO 91/18088; WO 93/09239; U.S. Pat. No. 4,797,368, U.S. Pat. No. 5,139,941, EP 488 528). These publications describe various AAV-derived constructs in which the rep and/or cap genes are deleted and replaced by a gene of interest, and the use of these constructs for transferring the said gene of interest in vitro (into cultured cells) or in vivo, (directly into an organism). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced are then purified by standard techniques.

The invention also relates, therefore, to an AAV-derived recombinant virus whose genome encompasses a sequence encoding a nucleic acid encoding a neuroactive substance flanked by the AAV ITRs. The invention also relates to a plasmid encompassing a sequence encoding a nucleic acid encoding a neuroactive substance flanked by two ITRs from an AAV. Such a plasmid can be used as it is for transferring the nucleic acid sequence, with the plasmid, where appropriate, being incorporated into a liposomal vector (pseudo-virus).

In a preferred embodiment, the vector is an adenovirus vector.

Adenoviruses are eukaryotic DNA viruses that can be modified to efficiently deliver a nucleic acid of the invention to a variety of cell types.

Various serotypes of adenovirus exist. Of these serotypes, preference is given, within the scope of the present invention, to using type 2 or type 5 human adenoviruses (Ad 2 or Ad 5) or adenoviruses of animal origin (see WO94/26914). Those adenoviruses of animal origin which can be used within the scope of the present invention include adenoviruses of canine, bovine, murine (example: Mav1, Beard et al., Virology 75 (1990) 81), ovine, porcine, avian, and simian (example: SAV) origin. Preferably, the adenovirus of animal origin is a canine adenovirus, more preferably a CAV2 adenovirus (e.g. Manhattan or A26/61 strain (ATCC VR-800), for example).

Preferably, the replication defective adenoviral vectors of the invention comprise the ITRs, an encapsidation sequence and the nucleic acid of interest. Still more preferably, at least the E1 region of the adenoviral vector is non-functional. The deletion in the E1 region preferably extends from nucleotides 455 to 3329 in the sequence of the AdS adenovirus (PvuII-BglII fragment) or 382 to 3446 (HinfII-Sau3A fragment). Other regions may also be modified, in particular the E3 region (WO95/02697), the E2 region (WO94/28938), the E4 region (WO94/28152, WO94/12649 and WO95/02697), or in any of the late genes L1-L5.

In a preferred embodiment, the adenoviral vector has a deletion in the E1 region (Ad 1.0). Examples of E1-deleted adenoviruses are disclosed in EP 185,573, the contents of which are incorporated herein by reference. In another preferred embodiment, the adenoviral vector has a deletion in the E1 and E4 regions (Ad 3.0). Examples of E1/E4-deleted adenoviruses are disclosed in WO95/02697 and WO96/22378, the contents of which are incorporated herein by reference. In still another preferred embodiment, the adenoviral vector has a deletion in the E1 region into which the E4 region and the nucleic acid sequence are inserted (see FR94 13355, the contents of which are incorporated herein by reference).

The replication defective recombinant adenoviruses according to the invention can be prepared by any technique known to the person skilled in the art (Levrero et al., Gene 101 (1991) 195, EP 185 573; Graham, EMBO J. 3 (1984) 2917). In particular, they can be prepared by homologous recombination between an adenovirus and a plasmid which carries, inter alia, the DNA sequence of interest. The homologous recombination is effected following cotransfection of the said adenovirus and plasmid into an appropriate cell line. The cell line which is employed should preferably (i) be transformable by the said elements, and (ii) contain the sequences which are able to complement the part of the genome of the replication defective adenovirus, preferably in integrated form in order to avoid the risks of recombination. Examples of cell lines which may be used are the human embryonic kidney cell line 293 (Graham et al., J. Gen. Virol. 36 (1977) 59) which contains the left-hand portion of the genome of an Ad5 adenovirus (12%) integrated into its genome, and cell lines which are able to complement the E1 and E4 functions, as described in applications WO94/26914 and WO95/02697. Recombinant adenoviruses are recovered and purified using standard molecular biological techniques, which are well known to one of ordinary skill in the art.

The instant invention demonstrates that efficient gene transfer into human neural progenitors can be obtained using recombinant adenoviruses. We have specifically shown that it is possibile to infect with an adenovirus encoding the Lac Z gene, proliferative precursors of neural cells derived from human embryos. We have furthermore developed conditions that have allowed us to obtain a large proportion of nervous cells to express the β-galactosidase marker gene both in vitro and in vivo after grafting. The efficiency of infection of the glial and neuronal lineages was particularly explored.

In a preferred embodiement, the invention therefore concerns human neural progenitor cells containing a recombinant adenoviral vector encoding a product of interest, such as a neuroactive substance.

Genetic modification of the cells or compositions according to the instant invention can also be made by chemical transfection. Suitable techniques include Ca phosphate precipitation, liposome-mediated transfection, cationic lipid transfection and lipopolyamine-mediated transfection.

Nucleic Acids

Genetic modification and grafting of the cells according to this invention now allows their use in numerous applications, depending on the introduced genetic material.

Reporter genes, such as the Lac Z gene, may help to solve important scientific questions in the field of neural development. In particular, the potential of progenitors explanted from various zones of the brain to survive and differentiate idependently of their origin could be investigated by following them after grafting in various zones of developing or adult brains.

Nucleic acids comprising a therapeutic gene are of particular interest. These genes include any gene encoding a neuroactive substance; a substance capable of exerting a beneficial effect on cells of the central nervous system. It may be a substance capable of compensating for a deficiency in or of reducing an excess of an endogenous substance. Alternatively, it may be a substance conferring new properties on the cells.

The neuroactive substance may be an antisense sequence or a protein. Among the proteins suitable for practice of the invention are growth factors, neurotrophic factors, cytokines, neurotransmitters, enzymes, neurotransmitter receptors and hormone receptors.

Preferably, the growth factor is a colony stimulating factor (G-CSF, GM-CSF, M-CSF, CSF, and the like), fibroblast growth factor (FGFa, FGFb) or vascular cell growth factor (VEGF). Among the neurotrophic factors, the preferred factors are ciliary neurotrophic factor (CNTF), glial cell maturation factors (GMFa, b), GDNF, BDNF, NT-3, NT-5 and the like. The complete nucleotide sequence encoding NT-3 is disclosed in WO91/03569, the contents of which are incorporated herein by reference.

Preferred cytokines are the interleukins and interferons. Enzymes included within the scope of the invention are the enzymes for the biosynthesis of neurotransmitters (tyrosine hydroxylase, acetylcholine transferase, glutamic acid decarboxylase) and the lysosomal enzymes (hexosaminidases, arylsulphatase, glucocerebrosidase, HGPRT). The enzymes involved in the detoxification of free radicals (super oxide dismutase I, II or III, catalase, glutathione peroxidase) are preferred. Receptors include the androgen receptors (involved in Kennedy's disease).

These proteins may be used in native form, or in the form of a variant or fragment thereof.

The neuroactive substance may also be an antisense sequence. The down regulation of gene expression using antisense nucleic acids can be achieved at the translational or transcriptional level. Antisense nucleic acids of the invention are preferably nucleic acid fragments capable of specifically hybridizing with a nucleic acid encoding an endogenous neuroactive substance or the corresponding messenger RNA. These antisense nucleic acids can be synthetic oligonucleotides, optionally modified to improve their stability and selectivity. They can also be DNA sequences whose expression in the cell produces RNA complementary to all or part of the mRNA encoding an endogenous neuroactive substance. Antisense nucleic acids can be prepared by expression of all or part of a nucleic acid encoding an endogenous neuroactive substance, in the opposite orientation, as described in EP 140308. Any length of antisense sequence is suitable for practice of the invention so long as it is capable of down-regulating or blocking expression of the endogenous neuroactive substance. Preferably, the antisense sequence is at least 20 nucleotides in length. The preparation and use of antisense nucleic acids, DNA encoding antisense RNAs and the use of oligo and genetic antisense is disclosed in WO92/15680, the contents of which are incorporated herein by reference.

The nucleic acid may be of natural or artificial origin. It may be especially genomic DNA (gDNA), complementary DNA (cDNA), hybrid sequences or synthetic or semisynthetic sequences. It may be of human, animal, plant, bacterial or viral origin and the like. It may be obtained by any technique known to persons skilled in the art, and especially by screening libraries, by chemical synthesis, or alternatively by mixed methods including chemical or enzymatic modification of sequences obtained by screening libraries. It is preferably cDNA or gDNA.

More preferred therapeutic products include in the case of Parkinson's disease the cDNA encoding tyrosine hydroxylase (TH) or a neurotrophic factor such as BDNF (brain derived neurotrophic factor) which favor the survival of dopaminergic neurons.

Similarly, for Alzheimer's disease, the cDNA encoding choline acetyl transferase and/or NGF (nerve growth factor) could prevent degeneration of cholinergic neurons.

Recent findings suggest that neurotrophic factors like BDNF and GDNF can be trophic factors for dopaminergic cells. Introduction into neural progenitors of genetic material encoding neurotrophic factors might in addition be of interest to improve graft survival.

Several adenovirus vectors encoding therapeutic genes have now been constructed. For instance, an adenovirus encoding tyrosine hydroxylase (TH) has been constructed. The grafting of in vitro infected neural cells according to the invention constitutes a very efficient way to deliver therapeutic amounts of TH in the brain. Other adenovirus-derived vectors encoding therapeutic genes include Ad-aFGF, Ad-bFGF, Ad-GDNF, Ad-GAD.

The genetic material of interest can also be an antisense-RNA or ribozyme or a DNA molecule encoding said antisense-RNA or ribozyme. These products are of particular interest for inhibiting production of toxic proteins such as β-amyloid precursor, TAU proteins, etc.

Preferably, the genetic material is a DNA encoding a protein or peptide of interest. As indicated above, said protein or peptide is preferably a neuroactive substance such as a growth factor (i.e. a cytokine) a neurotrophic factor, an enzyme or a neurotransmitter.

In an other embodiement, the genetic material is a DNA encoding an antisense-RNA or a ribozyme

Regulatory Regions

Generally, the nucleic acids of the present invention are linked to one or more regulatory regions. Said regions can include a regulatable or inducible promoter; neural cell-specific promoter, or viral promoter. Selection of the appropriate regulatory region or regions is a routine matter, within the level of ordinary skill in the art.

The regulatory regions may comprise a promoter region for functional transcription in neural progenitor cells, as well as a region situated in 3′ of the gene of interest, and which specifies a signal for termination of transcription and a polyadenylation site. All these elements constitute an expression cassette.

Promoters that may be used in the present invention include both constituitive promoters and regulated (inducible) promoters. The promoter may be naturally responsible for the expression of the nucleic acid. It may also be from a heterologous source. In particular, it may be promoter sequences of eucaryotic or viral genes. For example, it may be promoter sequences derived from the genome of the cell which it is desired to infect. Likewise, it may be promoter sequences derived from the genome of a virus, including the adenovirus used. In this regard, there may be mentioned, for example, the promoters of the E1A, MLP, CMV and RSV genes and the like.

In addition, the promoter may be modified by addition of activating or regulatory sequences or sequences allowing a tissue-specific or predominant expression (enolase and GFAP promoters and the like). Moreover, when the nucleic acid does not contain promoter sequences, it may be inserted, such as into the virus genome downstream of such a sequence.

Some promoters useful for practice of this invention are ubiquitous promoters (e.g. HPRT, vimentin, actin, tubulin), intermediate filament promoters (e.g. desmin, neurofilaments, keratin, GFAP), therapeutic gene promoters (e.g. MDR type, CFTR, factor VIII), tissue-specific promoters (e.g. actin promoter in smooth muscle cells), promoters which are preferentially activated in dividing cells, promoters which respond to a stimulus (e.g. steroid hormone receptor, retinoic acid receptor), tetracycline-regulated transcriptional modulators, cytomegalovirus immediate-early, retroviral LTR, metallothionein, SV-40, E1a, and MLP promoters. Tetracycline-regulated transcriptional modulators and CMV promoters are described in WO 96/01313, U.S. Pat. Nos. 5,168,062 and 5,385,839, the contents of which are incorporated herein by reference.

Pharmaceutical Administration

The present invention enables amplification and successful delivery of genes in vitro with high efficiency to human neural progenitor cells. These cells can then be grafted successfully in the brain of recipient organisms. Numerous neuroblasts expressing the transferred gene have been identified in such grafts. The invention thereby provides important clinical and scientific applications, such as treatment of neurodegenerative disorders.

The process according to the present invention enables one to target precisely a partiular region(s) of the brain, depending on the transferred therapeutic gene and the disorder to be treated. Thus, according to the site of the impairment to be treated, the administration is made into sites including, for instance, the striatum, hippocampus or substantia nigra. Preferably, they are grafted in the striatum.

According to the present invention, it is now possible, by stereotactic injection, to deliver a suspension of modified progenitor cells for engraftment. Determination of the coordinates for administration would be based on the disorder to be treated, and would be determined by the skilled practioner. The actual therapeutic regimen, including site of injection(s), number and schedule of injections, and particular dosage(s), would also be determined by the skilled practioner. In general, the number of cells engrafted at a site will be between 1×10³ and 1×10¹⁰, preferably 1×10⁵ to 1×10⁹, and more preferably 1×10⁶ to 1×10⁸.

The inventors have first established conditions enabling in vitro strong levels of expression in close to 100% of glial cells and more than 65% of neuroblasts without toxicity.

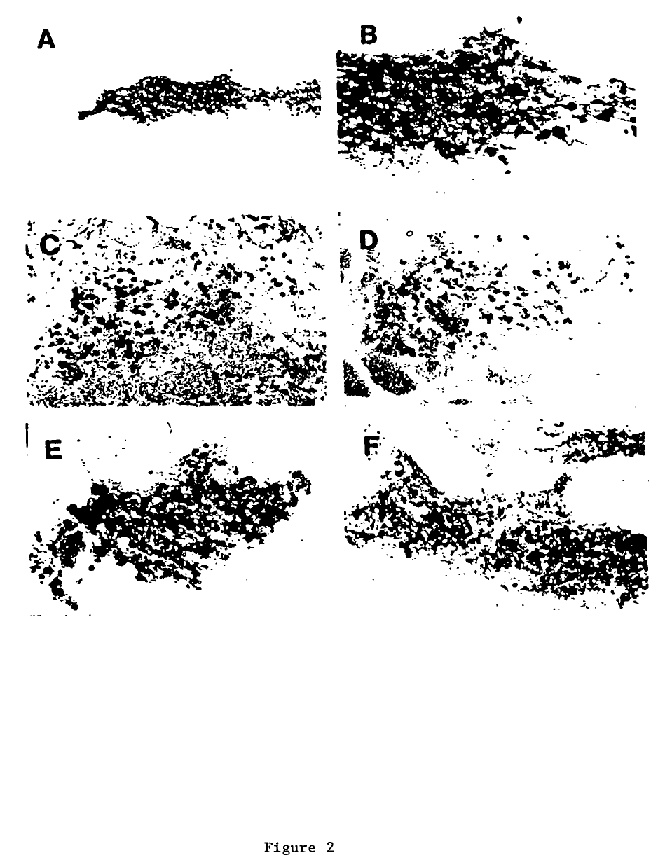

Cells infected under the best conditions were then engrafted. The inventors have unambiguously shown that cells present after transplantation are indeed human neuroblasts using specific markers. Absence of staining with anti-neurofilament indicated persistance of immature phenotype of the cells⁸. No human glial cells were identified in the grafts. Even if glial cells were a minority in the culture, as can be shown on the replating (FIGS. 2A & 2B), this result was unexpected. However, GFAP staining may be masked by diffusion in the cytoplasm of the β-galactosidase product, because of the high intensity of expression. Presence at the injection site of cells showing intense blue staining, unlabelled by neuronal markers (FIGS. 2E, 2F, 2G & 2H) seems to favour this hypothesis. Expression of the transgene was detected up to 3 weeks, the longest time tested so far.

Genetically-modified neural cells of the invention can be grafted in different location in the brain. More preferably, they are grafted in the striatum. Other sites include for instance hippocampus or substantia nigra. The grafting site depends on the transfered therapeutic gene and the disorder to be treated.

There are several important factors for ex vivo gene transfer to the brain, including 1) extent of expression; 2) stability of expression; 3) supply of material; 4) safety. Concerning the level of expression, the inventors have established conditions allowing the majority (>65%) of the cells to express β-galactosidase in vitro without toxicity. Survival of neural cells after grafting is a major concern of investigators in the field of intracerebral transplantation and the inventors have now observed large numbers of human neuroblasts expressing β-galactosidase in 3 out of 4 rats grafted with at least 10⁶ neural progenitors infected in vitro. In other experiments, survival was lower with grafts of 2×10⁵ cells and was not different whether the cells were infected or not. Injection of such high numbers of cultivated progenitors is probably necessary because of large-scale cell death immediately after grafting. In addition, we have shown that the density of the cells is important for survival and growth in vitro and that engraftment is highly improved where higher cell density is used. Furthermore the survival seems to be intrinsic to the grafting procedure since high survival yields are obtained throughout the course of the experiment and at the end upon replating the remaining cells. Moreover the survival is unaffected by genetic modification of the cells (i.e. adenoviral infection), as exemplified by the double-labelling with anti-NSE for neuroblasts or for all human cells by preliminary results of in situ hybridization with an oligonucleotide specific for the alu sequence, a human specific repetitive DNA. The number of surviving human cells expressing the reporter gene was high (7700±350 estimated in one rat). In the case of Parkinson's disease this number of cells expressing the tyrosine hydroxylase (TH) gene should be sufficient to compensate for the behavioral deficit in 6-OHDA lesioned hemiparkinsonian rats, since about 1600 dopaminergic neurons from human fetuses compensate the turning behavior induced by apomorphine.

Very interestingly, grafts continued to express the transgene 2 or 3 weeks post-grafting, the longest time tested. It is likely that long-term expression can be obtained since human neurons in vitro can express the reporter gene for up to 3 months and grafted astrocytes express β-galactosidase for up to 5 months.

It is also an object of the invention to provide an implant comprising a cell or composition as defined above. Preferably, the implant contains non-cellular material increasing survival and in vivo proliferation and differenciation of the cells. The implant can contain for instance collagen, gelatin, fibronectin, lectins, bio-compatible supports such as bone or polytetrafluoroethylen fibers, etc). The invention also concerns a method for the production of a therapeutic product in the brain of a recipient comprising grafting into the brain of said recipient a genetically-modified human neural progenitor cell containing introduced genetic material encoding said therapeutic product.

As mentioned previously, clinical improvement of Parkinson's disease requires today the implantation of mesencephalic dopaminergic cells from 3 to 4 human fetuses². Similarly, for certain neurological diseases such as Huntington's, evidence exists for the necessity to preserve the neuronal circuitry or replace it for therapeutical purposes which cannot be envisioned by direct injections of genes. For those reasons, the instant invention is of great interest in that it will now be possible to engraft numerous patients from a single fetus instead of one patient using 10 to 15 fetuses, as it is actually the case³. This largely obviate supply (and therefore some of the ethical) problems associated with the large numbers of human fetuses that would otherwise be required for the future development of restorative therapy in neurodegenerative diseases. In addition, the in vitro step to amplify the cells allows testing for the absence of contaminating agents such as viruses in the fetal tissue and thereby results in improved safety. The invention thereby provides safe, non toxic, long term expression of therapeutic genes in vivo. The invention is of particular interest in the treatment of neurodegenerative disorders such as neuropathies, strokes, spinal cord injury, amyotrophic lateral sclerosis, Huntington's chorea, Alzheimer's and Parkinson's diseases, cerebral palsy, epilepsia, lysosomal diseases (e.g. Tay Sachs and Sandhoff diseases, metachromatic leucodystrophy, Gaucher's disease, mucopolysaccharidosis, Lesh Nyhan, etc) as well as brain tumours.

### EXAMPLES

The present invention will be described in greater detail with the aid of the following examples which should be considered as illustrative and nonlimiting.

General Molecular Biology

The techniques of recombinant DNA technology are known to those of ordinary skill in the art. General methods for the cloning and expression of recombinant molecules are described in Maniatis (Molecular Cloning, Cold Spring Harbor Laboratories, 1982), and in Ausubel (Current Protocols in Molecular Biology, Wiley and Sons, 1987), which are incorporated by reference.

1. Generation and In Vitro Culture of Precursor Cells

Primary cultures of human fetal brain cells were initiated from human fetuses, obtained from legal abortions (Pr P. Blot & Pr J. F. Oury, Hopital R. Debré, Paris) after 5 to 12 weeks of gestation. Expulsion was done by seringe driven gentle aspiration under echographic control. Intact brains were obtained from 30 to 50% of the specimens. Fetuses collected in sterile hibernation medium⁹ were dissected in a sterile hood under a stereomicroscope (Wild). Brains were first removed in toto in hibernation medium containing penicillin G (500 U/ml, Specia), streptomycin (100 μg/ml, Diamant) and fungizon (5 μg/ml, Gibco BRL). For fetuses of 6 to 8 weeks the brain was separated into an anterior (telencephalic vesicles and diencephalon) and a posterior fraction (mesencephalon, pons and cerebellar enlage) and dissociated in toto after careful removal of meninges. For older fetuses, striatal, hippocampal, cortical and cerebellar zones expected to contain proliferative precursor cells were visualised under the stereomicroscope and dissected separately. Cells were transferred to either Opti MEM (Gibco BRL) containing 15% heat-inactivated fetal bovine serum (FBS) (Seromed), or to defined serum-free medium (DS-FM) with human recombinant bFGF (10 ng/ml, Boehringer), a minor modification of the Bottenstein-Sato medium¹⁰ with glucose (6 g/l), glutamine (2 mM, Gibco BRL), insuline (25 μg/ml, Sigma), transferrin (100 μg/ml, Sigma), sodium selenite (30 nM, Gibco BRL), progesterone (20 nM, Sigma), putrescine (60 mM, Sigma), penicillin G (500 U/ml), streptomycin (100 μg/ml) and fungizon (5 μg/ml). Cells (approximately 40000 per cm²) were grown at 37° C. in an atmosphere containing 10% CO₂ in tissue culture dishes (Falcon or Nunc) coated with gelatin (0.25% w/v) followed by matrigel (Gibco BRL), a basement membrane extract enriched in laminin and containing trace amounts of growth factors diluted 1 in 20. Cells were replated using trypsin-EDTA and were frozen in 10% dimethylsulphoxide in serum-free or serum-containing medium.

2. Genetic Modification of Neural Progenitor Cells

2.1. Use of Adenoviral Vectors

Adenoviral vectors represent efficient tools to transfer foreign genes to nerve cells as shown by recent studies where direct intracerebral injection to rodent brain has raised promises for gene therapy of central nervous system (WO94/08026). In order to amplify the number of cells suitable for grafting, the inventors investigated if recombinant adenoviruses can efficiently allow gene transfer to human neural progenitors. The inventors have now demonstrated the possibility of infecting with an adenovirus encoding the Lac Z gene, proliferative precursors of neural cells derived from human embryos. The inventors have furthermore developed conditions that have enabled one to obtain a large proportion of nervous cells to express the B-galactosidase marker gene both in vitro and in vivo after grafting.

2.1.1. Adenovirus Vectors

Many Adenovirus-derived vectors have been disclosed in the literature and can be prepared by the skilled man. Such vectors can be used in the present invention. (see in particular EP 185 573, Perricaudet et al., FEBS Letters 267 (1990) 60; Levrero et al, Gen 101 (1991) 195, FR 9305954, FR9308596, WO94/12649).

The Ad.RSVβgal vector has previously been disclosed in the literature. See for example Stratford-Perricaudet et a. (ref. 11). This vector contains the E. coli LacZ gene inserted in an adenovirus Ad5 deleted for the E1 and E3 regions.

2.1.2. Adenoviral Infection.

Human neuroepithelial stem cells were explanted from brains of human fetuses of 5 and 12 weeks of gestation, obtained after legal abortions. The human cells were amplified in vitro as described in example 1.

Cells seeded on 4 well dishes at a density of 2×10⁵ cells per well or on B6 at a density of 1×10⁶ cells per plate or on B10 at a density of 6 to 8×10⁶ cells per plate were infected with various MOI in respectively 300 μl, 1 ml and 3 ml of S-FDM. After one hour, respectively 300 μl, 1 ml and 3 ml of S-FDM were added to the plates and leaved for another 20 hours. Medium was then replaced with fresh one and cultures were grown until fixation with half the medium being replaced every 3 days.

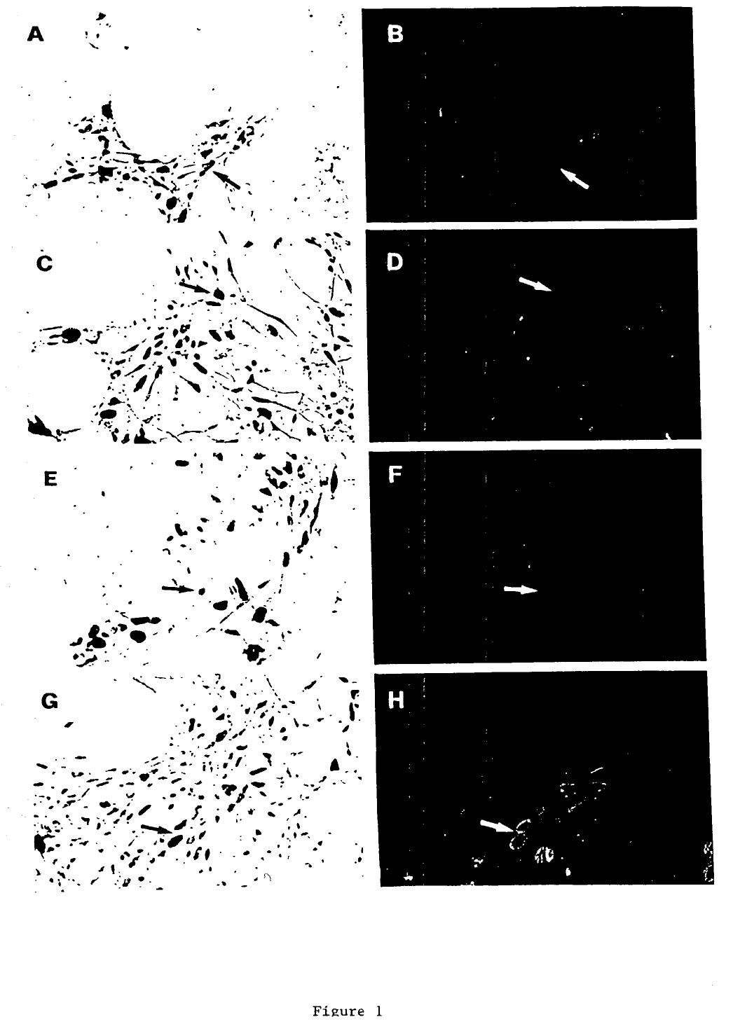

Adenoviral infections were performed with a replication-deficient adenovirus encoding the E. Coli Lac Z gene under the control of the RSV promoter, nuclearly targeted by the SV40 nuclear localization signal (Ad.RSVβgal) that has been previously described (see example 2.1.1.). In 4 independant experiments we observed β-galactosidase expression in more than 65% of the cells, 5 days after infection. All the cell types present in the cultures expressed the gene (FIG. 1, Table 1). Characterization of β-galactosidase expressing cells relies both on double staining experiments using specific immunocytochemical markers and the morphology of the cells (FIG. 1). Neuroepithelial stem cells are identified by their epithelial shape and their staining with anti-nestin¹² (FIGS. 1A & 1B) and vimentin¹³ (FIGS. 1C & 1D). Immature cells of the neuronal lineage, which we further refer as to neuroblasts, show rond refringent perikarya and bipolar processes together with expression of markers associated with early commitment to the neuronal lineage, MAP5¹⁴ and β3-tubulin¹⁵ (FIGS. 1E & 1F). The Absence of staining with anti-MAP2 and anti-neurofilament, which are expressed later in development attests for the immaturity of the cells⁸. Glial precursors harbor a flat morphology and are labelled by A2B5¹⁶ (FIGS. 1G & 1H) and HNK-1 while astrocytes show a typical morphology with protoplasmic-like processes and are decorated by anti-GFAP¹⁶. The β-galactosidase expressing cells, namely neuroepithelial stem cells (FIGS. 1A, B, C, D), immature cells of the neuronal lineage hereafter refered to as neuroblasts (FIGS. 1E, F), glial precursors (FIGS. 1G, H) or astrocytes (not shown) were characterized by double staining experiments using specific immunocytochemical markers and analysis of cell morphology. As described for rodents, β-galactosidase expression was stronger in glial than neuronal cell lineages: it was evident two days after infection only in glial cells (not shown) and at a low multiplicity of infection (MOI) (Table 1). Moreover, glial precursors were intensely blue, indicative of high level of activity (FIGS. 1G, H). No toxicity (severe cell damage leading to cell death) has been observed except at very high titers beginning around a MOI of 2000.

Close to 100% of the cells of the glial lineage show blue staining and about 65% neuroepithelial cells and neuroblasts expressed the reporter gene (Table 1).

TABLE 1
Estimation of the percentage of human cells from the neuronal or
glial lineages expressing β-galactosidase after Ad-RSVβgal infection

| | % 0f double-labelled | | | |
|---|---|---|---|---|
| | cells for MOI (pfu/cell) of | | | |
| Cell lineage | 1 | 10 | 100 | 500 |
| Neuronal (β₃-tublin+/β-gal+) | 0 | <1 | 50 | 65 |
| Glial (GFAP + /β-gal+) | 4 | 45 | 90 | 99 |

Cells were grown in DS-FM containing 10 ng/ml of bFGF for 13 days in vitro (DIV) before infection at various MOI. X-gal staining and immunocytochemistry were performed 5 days later as indicated in examples 3.1. and 3.2. Expression of β3 tubulin and GFAP were considered to be indicative of cells of the neuronal and glial lineages respectively.

2.2. Use of Other Vectors

As indicated above, other types of vectors can be used to genetically modify the neural progenitors according to the invention. This can be viral or non-viral (chemical) vectors. Preferred viral vectors include AAV, retroviruses, herpes viruses and vaccinia virus. Non viral vectors include Calcium-phosphate precipitation, liposome-mediated transfection, cationic lipid transfection and lipopolyamine-mediated transfection.

3. Intracerebral Grafting.

We tested whether neural progenitors infected in vitro survive after grafting. To amplify the number of cells before infection and grafting, human neuroepithelial cells explanted from the cortex of a 12 week fetus were grown for 4 days in serum-containing medium then for an additional 7 days in defined serum-free medium. These conditions were chosen to favor immature precursors of the neuronal lineage. The cultures were infected at a MOI of 500 to maximize expression in neuroblasts and the following day various numbers of infected cells, ranging from 0.3 to 1.5×10⁶, were implanted in the striatum of immunosuppressed rats. More specifically, twenty hours after exposure to the virus, the cells were rinsed with trypsin-EDTA, then incubated in the same medium for 5 min at 37° C. OptiMEM containing 15% FBS was added and the cells were detached from the dishes and centrifuged at 1000 rpm for 10 nm. Cells were resuspended in DS-FM, counted, centrifuged again, resuspended in DS-FM at the desired density and kept on ice throughout the grafting session. Thirteen adult female Sprague-Dawley rats (Iffa-Credo) were engrafted under anaesthesia with equitesin (3 ml/kg). Numbers of cells grafted were 3×10⁵ in 2 rats, 4×10⁵ in3 rats, 6×10⁵ in 4 rats, 1×10⁶ in 2 rats and 1.5×10⁶ in 2 rats. 1.5 to 3 μl of the cell suspension was stereotactically injected using 10 μl Hamilton syringes into the striatum at the following coordinates (tooth bar fixed at 0), namely +1.2 anterior to the bregma, 2.6 lateral to midline and 4.5 ventral to the dural surface intraperitonally. Animals were injected daily with cyclosporin (Sandoz) at 10 mg/kg and oxytetracycline (Sigma) was provided in the drinking water to prevent infections. Their fate was examined 2 or 3 weeks after grafting. Large numbers of blue cells clustered at the injection site were observed in 4 out of 13 rats (FIG. 2). Importantly, 3 out of 4 rats grafted with 1 or 1.5×10⁶ cells (density: 5×10⁵ cells/μl) displayed high numbers of blue cells while only one out of 9 rats grafted with lower numbers did contain surviving cells.

We therefore assessed the viability of the infected cells after harvesting and before grafting. The percentage of viable cells, kept in test tube or passed through the seringe needle, was counted several times using the trypan blue exclusion technique. The viability was around 85% throughout the grafting session. Moreover, ungrafted cells were replated and grown in serum-free medium for another 5 days before fixation and X-gal staining: 65% of the cells expressed 13-galactosidase. Thus, the loss of the grafted cells in 9 rats could not be explained by a poor viability of the cells. Presumably, it results from a post-grafting event. Detailed analysis in one animal revealed that the number of cells expressing β-galactosidase in the graft after 3 weeks was estimated to be 7700±350. Of the 1×10⁶ grafted cells, 6.5×10⁵ cells were estimated to express the transgene. Thus at least 1.2% of the human cells had survived after grafting.

We verified the specificity of the X-gal staining, because long incubations can reveal endogeneous β-galactosidase in blood vessels or in macrophages. X-gal staining was compared to that of an antibody specific for the E. coli β-galactosidase on adjacent sections of one grafted brain. The labeling patterns were similar (FIGS. 2C, D). That the blue cells were human was further confirmed (for neuronal lineage) by staining with an antibody specific for the human neuron specific enolase¹⁷ (NSE) (FIGS. 2A, B). Moreover, preliminary results of in situ hybridization with an oligonucleotide specific for a human specific repetitive DNA, the alu sequence, confirmed that all blue cells were of human origin.

Surviving grafted cells were further identified by double staining experiments. Numerous cells were characterized as neuroblasts by labeling with specific markers: anti-NSE (FIGS. 2A, B), anti-β3tubulin¹⁵ (FIG. 2E), anti MAP5¹⁴ (FIG. 2F). Absence of staining with anti-neurofilament indicated persistance of an immature phenotype (cells of their age would be immature in vivo). In contrast, there was no double-labeling in grafts with anti-GFAP¹⁶ (FIG. 2C), anti-vimentin or A2B5¹⁶. This is consistent with the fact that glial cells represented a minority of the cells used (not shown).

3.1. β-Galactosidase Histochemistry.

At various times after grafting, animals were perfused under chloral hydrate anesthesia with 0.9% saline followed by ice cold 4% paraformaldehyde in phosphate buffered saline (pH 7.4) (PBS) over 7 min at 40 m1 min. Brains were removed and postfixed in the same solution and stored in 20% sucrose, PBS for few days before obtaining 15 μm sections with a cryostat or 40 μm microtome sections. Cultures were fixed in PBS containing 4% paraformaldehyde. Slides or cultures were incubated in X-gal reaction mixture containing 35 mM K₃Fe(CN)₆, 35 mM K₄Fe(CN)₆.3H₂O, 2 mM MgCl₂, 0.01% sodium deoxycholate, 0.02% Nonidet-P40 and 1 mg/ml 5-bromo-4-chloro-3-indolyl-β-D-galactopyranoside (X-gal, Uptima, dissolved at 40 mg/ml in N-N-dimethylformamide and kept at −20° C.) in PBS (pH 7.4) for 3 to 18 h at 37° C. Blue cells were counted in a serie of sections (one in each four consecutive sections) according to Abercrombie⁴ (estimated size of nuclei: 7 μm).

3.2. Immunohistochemistry.

Cells or slides were processed for β-galactosidase histochemistry then for immunohistochemistry using standard techniques. Primary antibodies included: polyclonal rabbit anti-nestin 129, a gift from Pr R. D. G. McKay, anti-β-galactosidase (Cappel), mouse monoclonal: A2B5, a gift from Dr C. Gouget-Zalc, HNK1, a gift from Pr J. R. Sanes, anti-neurofilament pool, a gift from Pr D. Paulin, GA5 and anti-vimentin (DAKO), anti-β3tubulin and anti-MAP5 (Sigma), anti-MAP2 (Boehringer), Anti-NSE, human specific (Monosan). Secondary antibodies and revelation systems were: texas red conjugated anti-rabbit IgG (Vector), FITC-conjugated anti-mouse IgM (Sigma), the vectastain kit for rabbit IgG (Vector), biotinylated anti mouse Ig and IgM and the avidin-biotin-streptavidin complex (Amersham).

All the references discussed herein are incorporated by reference.

One skilled in the art will readily appreciate the present invention is well adapted to carry out the objects and obtain the ends and advantages mentioned, as well as those inherent therein. The peptides, polynucleotides, methods, procedures and techniques described herein are presented as representative of the preferred embodiments, and intended to be exemplary and not intended as limitations on the scope of the present invention. Changes therein and other uses will occur to those of skill in the art which are encompassed within the spirit of the invention or defined by the scope of the appended claims.

### REFERENCES

1. Lindvall, O. et al. Science 247, 574-577 (1990).

2. Lindvall, O. et al. Ann.Neurol. 31, 155-165 (1992).

3. Björklund, A. Nature 362, 414-415 (1993).

4. Abercrombie, M. Anat.Rec. 94, 239-247

5. Reynolds, B. A. & Weiss, S. Science 255, 1707-1710 (1992).

6 Cattaneo, E. & McKay, R. Nature 347, 762-765 (1990).

7. Ray, J. et al., Proc. Natn. Acad. Sci. U.S.A. 90, 3602-3606 (1993).

8. Tohyama et al., J.Comp.Neurol. 310, 285-299 (1991).

9. Brundin, P. Neural transplantation, apractical approach. S. Dunnnett & A. Björklund, ed., 8, 139-160. IRL Press, Oxford 1992.

10. Bottenstein, J. E. & Sato, G. H. Proc. Natn. Acad. Sci. U.S. 76, 514-517 (1979).

11. Stratford-Perricaudet et al., J. Clin. Invest. 90, 626-630 (1992).

12. Frederiksen, K. & McKay, R. J Neurosci. 8, 1144-1151 (1988).

13. Stagaard, M. & Mollgard, K. Anat. Embryol. 180, 17-28 (1989).

14. Tucker, R. P. Brain Res.Rev. 15, 101-120 (1990).

15. Denoulet et al., Gene 50, 289-297 (1986).

16. Raff, M. C. Science 243, 1450-1454 (1989).

17. Kato et al., Neurochem. Int. 6, 51-54 (1984).

1. A human neural progenitor cell comprising an exogenous nucleic acid encoding a neuroactive substance.

2. A human neural progenitor cell according to claim 1, which is reactive with anti-nestin and anti-vimentin antibodies.

3. A human neural progenitor cell according to claim 1, wherein said cell is derived from human foetal brain.

4. A human neural progenitor cell according to claim 1, wherein said cell is a neuroepithelial stem cell.

8. A human neural progenitor cell according to claim 1, wherein said nucleic acid has been introduced into said cell with a viral vector.

9. A human neural progenitor cell according to claim 8, wherein said viral vector is selected from the group consisting of adenovirus, Herpes virus, AAV, retrovirus and vaccinia virus.

10. A human neural progenitor cell according to claim 9, wherein said viral vector is a replication defective adenoviral vector.

11. A human neural progenitor cell according to claim 1, wherein said nucleic acid has been introduced into said cell by calcium-phosphate precipitation, liposome-mediated transfection, cationic lipid transfection, or lipopolyamine-mediated transfection.

12. A human neural progenitor cell according to claim 1, wherein said nucleic acid is DNA or RNA.

13. A human neural progenitor cell according to claim 12, wherein said nucleic acid is a DNA encoding a protein or peptide.

14. A human neural progenitor cell according to claim 13, wherein said protein or peptide is selected from the group consisting of growth factors, neurotrophic factors, and enzymes.

15. A human neural progenitor cell according to claim 12, wherein said nucleic acid is a DNA encoding an antisense-RNA or a ribozyme.

16. A human neural progenitor cell according to claim 1, wherein said nucleic acid is operably linked to a regulatory region.

17. A human neural progenitor cell according to claim 1, wherein said regulatory region comprises a regulatable promoter, an inducible promoter, a neural cell-specific promoter or a viral promoter.

18. A human neural progenitor cell comprising a replication defective adenovirus comprising a nucleic acid encoding a neuroactive substance.

19. An implant comprising a population of cells according to claim 1.

20. A composition comprising human neural progenitor cells comprising an exogenous nucleic acid encoding a neuroactive substance.

21. A composition according to claim 20, further comprising neuroblasts comprising an exogenous nucleic acid encoding a neuroactive substance.

22. A composition according to claim 20, further comprising glial precursors comprising an exogenous nucleic acid encoding a neuroactive substance.
